(19) United States
(12) Patent Application Publication
(10) Pub. No.: US 2025/0010073 A1
(43) Pub. Date: Jan. 9, 2025

(54) METHOD OF MEASURING THE CONTRIBUTION OF EACH CHANNEL WITHIN A COCHLEAR IMPLANT TO SPEECH RECOGNITION ACCURACY ON A PER-PATIENT BASIS

(71) Applicant: Father Flanagan's Boys' Home Doing Business as Boys Town National Research Hospital, Omaha, NE (US)

(72) Inventor: Adam Bosen, Omaha, NE (US)

(21) Appl. No.: 18/762,011

(22) Filed: Jul. 2, 2024

Related U.S. Application Data
(60) Provisional application No. 63/512,140, filed on Jul. 6, 2023.

(51) Int. Cl. A61N 1/36

(52) U.S. Cl. CPC A61N 1/36039

(57) ABSTRACT

To address issues for cochlear implant users relating to speech recognition, a solution includes linking speech cue access to properties of the listener's (i.e., cochlear implantee's) auditory nerve and cochlear implant processor. The information can be used to determine differences in implantee's ability to extract speech cues from different channels of the speech spectrum. The information can then be used in clinical assessments and interventions to more precisely target channel-specific impairments to improve the implantee's auditory benefits from the cochlear implants.

### CROSS REFERENCE TO RELATED APPLICATIONS

This application claims priority under 35 U.S.C. § 119 to provisional patent application U.S. Ser. No. 63/512,140, filed Jul. 6, 2023. The provisional patent application is herein incorporated by reference in its entirety, including without limitation, the specification, claims, and abstract, as well as any figures, tables, appendices, or drawings thereof.

### TECHNICAL FIELD

The present disclosure relates generally to the auditory field, and more particularly to the field of cochlear implants. More particularly still, but not exclusively, the disclosure is directed towards systems and methods to attempt to improve the functionality of cochlear implants on a per user basis by way of a test that can quickly and easily determine features of an implanted cochlear implant that could be modified to improve hearing and speech recognition for the user.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

A cochlear implant (CI) is a surgically implanted neuroprosthesis that provides a person who has moderate-to-profound sensorineural hearing loss with sound perception. With the help of therapy, cochlear implants may allow for improved speech understanding in both quiet and noisy environments. A CI bypasses acoustic hearing by direct electrical stimulation of the auditory nerve. Through everyday listening and auditory training, cochlear implants allow both children and adults to learn to interpret those signals as speech and sound.

The implant has two main components. The outside component is generally worn behind the ear, but could also be attached to clothing, for example, in young children. This component, the sound processor, contains microphones, electronics that include digital signal processor (DSP) chips, battery, and a coil that transmits a signal to the implant across the skin. The inside component, the actual implant, has a coil to receive signals, electronics, and an array of electrodes which is placed into the cochlea, which stimulate the cochlear nerve.

From the early days of implants in the 1970s and the 1980s, speech perception via an implant has steadily increased. More than 200,000 people in the United States had received a CI through 2019. Many users of modern implants gain reasonable to good hearing and speech perception skills post-implantation, especially when combined with lipreading. One of the challenges that remain with these implants is that hearing and speech understanding skills after implantation show a wide range of variation across individual implant users. Factors such as age of implantation, parental involvement and education level, duration, and cause of hearing loss, how the implant is situated in the cochlea, the overall health of the cochlear nerve, but also individual capabilities of re-learning are considered to contribute to this variation.

Only about 25% of post-lingually deafened individuals with cochlear implants achieve word recognition accuracy above 80%. The majority have worse outcomes, with an average word recognition accuracy of around 50%, and some individuals unable to recognize speech at all. This lack of consistent benefit from implantation is a substantial failure for the individuals at the lower end of this range, because they underwent a costly and invasive surgery only for the expected benefits to remain unrealized. Thus, there is a critical need for individualized interventions to help patients with poor speech recognition outcomes.

The reason that there is such a large range of outcomes post-implantation is because the many mechanisms that can impair transmission of speech information through the auditory pathway are heterogeneous across patients and across place within an implanted ear. Cochlear implants primarily convey amplitude envelope cues, which carry crucial information for speech perception. Consequently, speech recognition outcomes are predicted by the extent to which the listener's auditory pathway can encode changes in the envelope across place and over time. Heterogeneity in the precision with which cues are represented in the auditory pathway manifests as variable sensitivity to spectral and temporal envelope cues for speech recognition across individuals. Additionally, encoding of these cues can also vary dramatically across place within an ear. Thus, individualized assessments need to be capable of identifying which mechanisms are impairing transmission of speech cues throughout each patient's cochlea.

Several methods have been developed for measuring how well the amplitude envelopes that convey speech cues are represented in the auditory pathway, including behavioral thresholds, electrophysiological recording, and medical imaging. Typically, these methods are used to identify locations within the cochlea that have undesirable properties and then disable or modify the electrical stimulation provided to those locations. Studies using this approach usually show improvements in speech recognition accuracy on average, but some individuals show negligible benefit or even reductions in speech recognition accuracy following intervention. This lack of consistent benefit indicates that these assessments will sometimes fail to identify the critical mechanisms that impair cue transmission.

Thus, there exists a need in the art for systems and/or methods to identify issues related to cochlear implants and speech cues to improve the functionality of the cochlear implants and to maximize the benefit that every patient receives from their cochlear implant. There is also a need for methods to identify issues that vary across patients.

### SUMMARY

The following objects, features, advantages, aspects, and/or embodiments are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

It is a further object, feature, and/or advantage of any of the aspects of any of the embodiments of the present disclosure to identify the properties of the auditory nerve and cochlear implant processor that are needed to convey speech cues within each channel. This information will provide criteria for clinical decision making and device design that will enable optimization of access to speech cues for all patients.

It is still yet a further object, feature, and/or advantage of any of the aspects of any of the embodiments of the present disclosure to identify auditory nerve properties that impart encoding of speech cues.

It is still yet a further object, feature, and/or advantage of any of the aspects of any of the embodiments of the present disclosure to determine whether bilateral interactions reduce access to speech cues within channels.

It is still yet a further object, feature, and/or advantage of any of the aspects of any of the embodiments of the present disclosure to examine effects of listening conditions on access to speech cues.

It is still yet a further object, feature, and/or advantage of any of the aspects of any of the embodiments of the present disclosure to identify the electrical stimulus features that convey speech cues.

The systems and/or methods disclosed herein can be used in a wide variety of applications. For example, while many of the features will be beneficial to cochlear implant users, it should be appreciated that the same could be used for any number of auditory issues relating to nerves and how they interact with neuroprosthetic devices.

The systems provided can be incorporated into systems or kits which accomplish some or all of the previously stated objectives. This can include any number of clinical devices used with assessing the efficacy of neuroprosthetic devices, such as cochlear implants.

According to at least some aspects of the disclosure, a method for identifying channel importance of a cochlear implant user comprises providing an audio example to the cochlear implant user; masking one or more channels of the cochlear implant during the audio example, wherein a masker manipulates the availability of information available from the audio example on the channel to obtain a target-to-masker ratio; and identifying channel importance by determining the fixed effect of the target-to-masker ratio within each channel on a recognition accuracy of the audio example.

According to at least some aspects of some embodiments, the audio example comprises: a verbal word; or an audio file played on a speaker.

According to at least some aspects of some embodiments, the method further comprises having the user identify the content of the audio example to determine the recognition accuracy.

According to at least some aspects of some embodiments, the identifying of the audio example is done verbally, in writing, or via multiple choice.

According to at least some aspects of some embodiments, the step of masking the channel comprises masking each channel with a pure tone at a middle of that channel's frequency range.

According to at least some aspects of some embodiments, the method further comprises deactivating channels of the cochlear implant for channels having low channel importance.

According to at least some aspects of some embodiments, the step of masking the channel comprises pseudorandomly selecting the masking level for each channel.

According to additional aspects, a method of improving the performance of a cochlear implant in a user comprises identifying channel importance for an implanted cochlear implant of the user by masking channels of the cochlear implant; based on the identified channel importance, adjusting the cochlear implant channels.

According to at least some aspects of some embodiments, the adjusting the cochlear implant channels comprises deactivating channels having low channel importance.

According to at least some aspects of some embodiments, the step of identifying channel importance comprises: providing an audio example to the cochlear implant user; masking one or more channels of the cochlear implant during the audio example, wherein a masker manipulates the availability of information available from the audio example on the channel to obtain a target-to-masker ratio; and identifying channel importance by determining the fixed effect of the target-to-masker ratio within each channel to an audio recognition accuracy of the audio example.

According to at least some aspects of some embodiments, the audio example comprises: a verbal word; or an audio file played on a speaker.

According to at least some aspects of some embodiments, the method further comprises having the user repeat the audio example to determine the audio recognition accuracy.

According to still additional aspects of the disclosure, a method for identifying channel importance of a cochlear implant user, comprises providing an audio example to the cochlear implant user; filtering one or more frequency bands that correspond to one or more channels of the cochlear implant; and identifying channel importance by determining which of the one or more filtered channels affects the cochlear implant user's hearing.

According to at least some aspects of some embodiments, the filtering one or more frequency bands comprises manipulating a presence of speech information within a channel associated with the audio example.

According to at least some aspects of some embodiments, the filtering one or more frequency bands comprises manipulating an absence of speech information within a channel associated with the audio example.

According to at least some aspects of some embodiments, identifying channel importance comprises a fixed effect of filtering out each channel on recognition accuracy in a generalized linear model with a logistic link function.

According to at least some aspects of some embodiments, filtering one or more frequency bands measures an impact of removing each channel on word recognition accuracy.

According to still additional aspects of the disclosure, a method for identifying channel importance of a cochlear implant user comprises providing an audio example to the cochlear implant user; manipulating one or more channels of the cochlear implant during the audio example; and identifying channel importance by determining which of the one or more manipulated channels affects the cochlear implant user's hearing.

According to at least some aspects of some embodiments, the step of manipulating the one or more channels of the cochlear implant comprises masking one or more channels of the cochlear implant during the audio example, wherein a masker manipulates the availability of information available from the audio example on the channel to obtain a target-to-masker ratio.

According to at least some aspects of some embodiments, the step of manipulating the one or more channels of the cochlear implant comprises filtering one or more frequency bands that correspond to one or more channels of the cochlear implant.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Several embodiments in which the present disclosure can be practiced are illustrated and described in detail, wherein like reference characters represent like components throughout the several views. The drawings are presented for exemplary purposes and may not be to scale unless otherwise indicated.

FIG. 1 includes graphs showing the average importance (in black) of each channel for speech recognition in a cochlear implant simulation using a vocoder and in individuals with cochlear implants made by two different companies. Channel importance for individuals are shown in color.

FIG. 2 is a schematic showing an audio example used to measure channel importance, which is the change in speech recognition accuracy that results from varying pure tone masker levels in each channel.

FIG. 3 is a schematic showing how channel fidelity is controlling by manipulating target-to-masker ratio in each channel.

FIG. 4 is a graph showing the relationship between speech recognition accuracy and fidelity for channels with high (black) or low (gray) importance.

FIG. 5 is a graph showing how channel importance varies across channels in a Med-El device user with 11 active channels using the methods disclosed herein and depicted in FIG. 2.

FIG. 6 is a schematic showing another audio example used to measure channel importance by controlling channel audibility by filtering speech to include or exclude the frequency range corresponding to each channel.

FIG. 7 is a graph showing how channel importance varies across channels in an Advanced Bionics device user with 13 active channels using the methods disclosed herein and depicted in FIG. 6.

FIG. 8 is a graph showing outliers in channel importance relative to the group average for that channel.

An artisan of ordinary skill in the art need not view, within isolated figure(s), the near infinite distinct combinations of features described in the following detailed description to facilitate an understanding of the present disclosure.

### DETAILED DESCRIPTION

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refers to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variables, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

As will be understood, any of the aspects of any of the embodiments disclosed herein address the limitations of previous work by using a novel method to quantify the overall transmission of speech cues across place within each ear, which is herein referred to as channel importance. Measuring channel importance enables the identification of the impact of multiple mechanisms that can impair information transmission, which in turn enables prioritizing the diagnosis and treatment of mechanisms with large impacts on speech recognition outcomes.

Cochlear implants divide the auditory spectrum into discrete bands and transduce the acoustic amplitude envelope within each band to an amplitude modulated electrical pulse train emitted by an electrode at a roughly corresponding place within the cochlea. This division of the auditory spectrum yields an array of channels within the implant, with each channel conveying some information about the acoustic input. Channel importance—the amount of speech information that each channel conveys—is determined by the availability of acoustic information in the input, the transduction of that information into electrical pulses, and the encoding of that electrical waveform in the response of the auditory nerve.

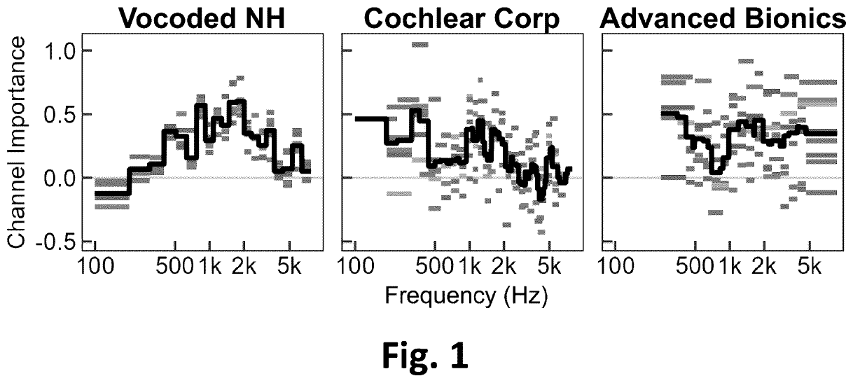

Channel importance is assessed by removing or reducing the availability of information within a channel and measuring the resulting change in speech recognition accuracy. Prior assessments of channel importance found that channels with frequency ranges below 400 Hz and around 1.2 kHz have the highest average importance in listeners with cochlear implants. However, channel importance substantially deviates from the mean in many individuals, as shown in FIG. 1. The ideal outcome is when channels have high importance relative to the group, which indicates that the cochlear implant user can extract most of the speech information that is present in that channel. Below the upper edge, the amount of information conveyed by the channel is impaired, with some channels contributing little or even impeding speech recognition when importance is negative.

As shown in FIG. 1, the importance of each channel in a cochlear implant for speech recognition varies across ears much more than in listeners with normal hearing (NH). Colored horizontal lines show the importance of each channel in one ear, and the thick black line shows average importance across frequencies. Many listeners with cochlear implants show little ability to extract speech information from channels that are important on average, which limits overall speech recognition outcomes. The figure shows data for (A) normal hearing listening through a vocoder that simulates hearing with a cochlear implant; (B) users of cochlear implants from Cochlear Corporation; and (C) users of cochlear implants from Advanced Bionics Corporation.

This variability across listeners demonstrates a need to consider two factors: how much information a listener is extracting from a channel, and how much information they could be extracting. For example, there is one listener in the Cochlear Corp group and another in the Advanced Bionics group in FIG. 1 who have low importance for their lowest frequency channel (˜200-400 Hz) and are outliers relative to their group. For these listeners, there is likely some impairment that is limiting access to speech cues in that channel and could be resolved via modification of their clinical program. In contrast, none of the listeners who use Cochlear Corporation devices extract much information from frequencies above 6 kHz. Thus, while speech cues conveyed by channels with frequencies above 6 kHz may be poorly conveyed in some listeners, it is unlikely that modifying their clinical program would improve speech recognition outcomes, and instead some feature of the device or processing strategy needs to be revised to improve access to speech cues at high frequencies.

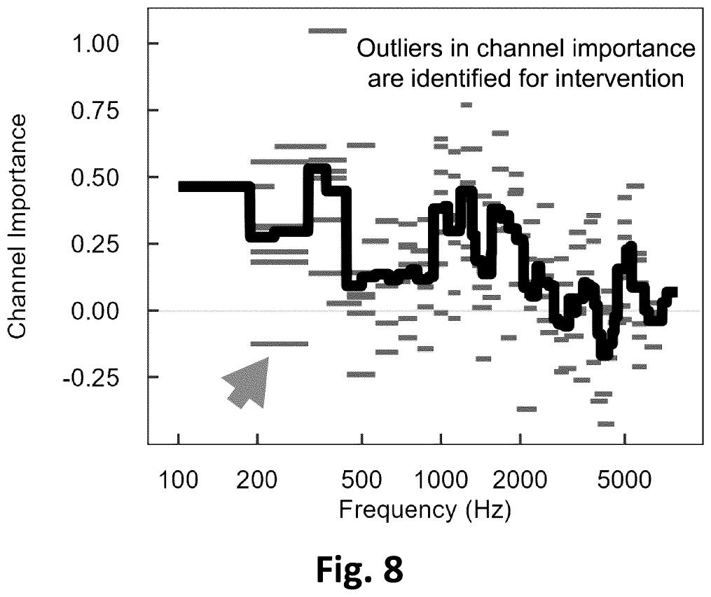

Targeted intervention to improve the amount of speech information transmitted per channel requires knowing which channel properties critically limit importance (see, e.g., FIG. 8 for depiction of outliers in channel importance). According to at least some aspects of some embodiments of the present disclosure, systems and/or methods disclosed will identify key properties of the neural response to electrical stimulation, cochlear implant processing strategy, and the contents of the acoustic input that determine channel importance. Estimates of channel importance bridge a vital gap between whole-spectrum assessments of speech recognition and channel-specific psychophysical and electrophysiological assessments of spectrotemporal resolution. Decades of prior work have proposed and developed assessments of several channel-specific candidate mechanisms that substantially limit the transmission of speech cues, but without a link between those assessments and individual differences in channel importance for speech recognition it is difficult or impossible to determine which mechanisms have the greatest impact on outcomes with cochlear implants. Identifying the relative impact of these mechanisms on speech recognition will enable users to prioritize those mechanisms for individualized clinical assessment and intervention. In addition, average channel importance in listeners with cochlear implants differs from average band importance in listeners with acoustic hearing, so clinical practice would benefit from knowing which channels have high potential importance to prioritize assessment and intervention for those channels.

Additional or the same aspects of the present disclosure will form the basis for clinical implementation of several tools for individualized assessment and intervention to improve speech recognition outcomes in subsequent work. Measures of channel importance can be used to inform interpretation of electrophysiological assessments of neural encoding. Such assessments require no engagement from a patient, can be conducted with standard audiological equipment, and can be completed in ˜10 minutes, which makes this form of assessment feasible in many clinics. Intelligibility metrics that incorporate patient-specific neural encoding will facilitate the design of novel processing strategies and individualized selection of optimal strategies. Identifying effects of stimuli, hearing configuration, and listening condition on channel importance across will ensure that assessments of importance inform interventions that yield real-world gains in speech recognition outcomes.

As is understood, the present disclosure provides novel measures of the importance of each channel for speech recognition in patients with cochlear implants. While speech recognition is an active area of research in this population and heterogeneity in speech outcomes is well documented, there has been few to no studies which has examined individual differences in channel-specific contributions to speech recognition. This methodological innovation of the present disclosure is a critical advancement over current approaches because it enables assessment of the mechanisms that limit place-specific speech cue transmission on an individual basis without averaging across participants or assuming that channel importance does not vary across place.

The following is an example of an approach covering aspects and/or embodiments of the novel systems and/or methods of the present disclosure. For example, the following provides at least one way to test a cochlear implant user's ability to identify sounds based on the channels of the implant, which may provide insight into issues related to the ability to hear, and also may provide a pathway for improving the experience of the implant user.

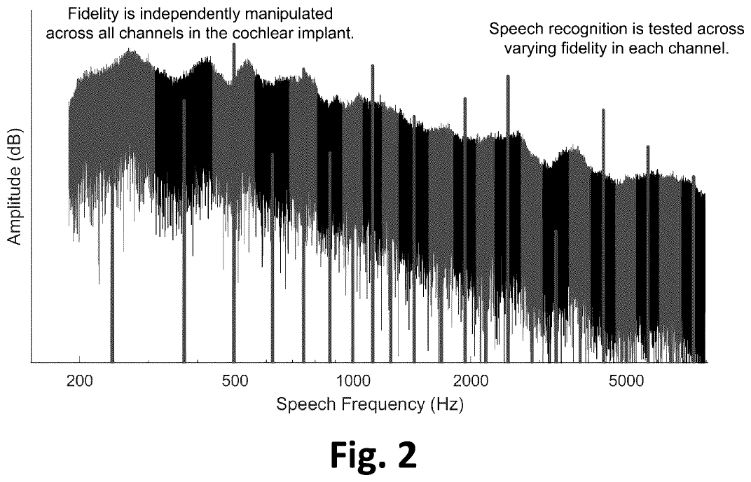

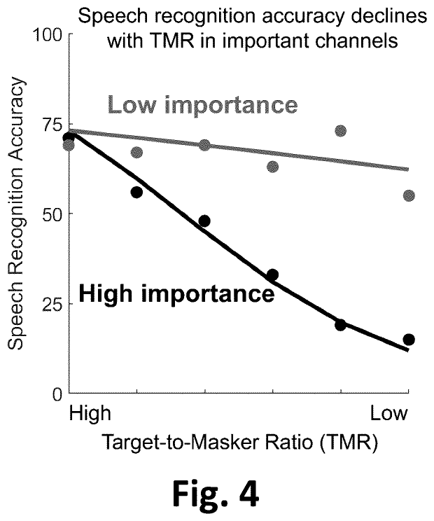

As will be understood, FIGS. 2-4 and 6 depict aspects associated with at least one embodiment of the present disclosure. The figures are provided as support for the methods and/or systems that can be used as part of the disclosure but should not be limiting on the overall disclosure as a whole.

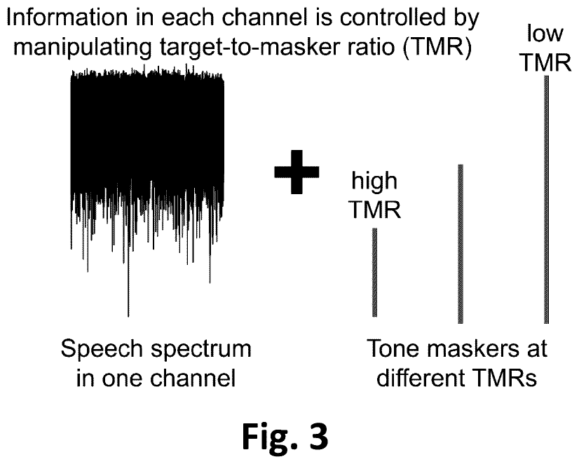

According to at least some aspects of some embodiments, participants (i.e., cochlear implant users) hear and repeat aloud monosyllabic words in the presence of a tone complex masker (see, e.g., FIG. 2). The masker is designed to systematically manipulate the availability of speech information within each channel across words, which allows us to measure the impact of masking each channel on word recognition accuracy (see, e.g., FIG. 3). Each channel is masked with a pure tone at the middle of that channel's frequency range, and the amplitude of each tone will be varied across words to independently manipulate the target-to-masker ratio (TMR) across channels. TMR will range of +30 to −10 dB. Centering the average TMR at +10 dB minimally affects overall recognition accuracy, and the extents of the range roughly match the variability in in-channel energy across words. The TMR range is pseudorandomly sampled such that the TMR of each channel is independent across words. Tone phase is selected to minimize the temporal envelope fluctuation of the tone complex. To facilitate attention to target words, a carrier word (“ready”) is presented before each target word and the masker starts one second before the onset of the carrier and ends one second after the offset of the target word.

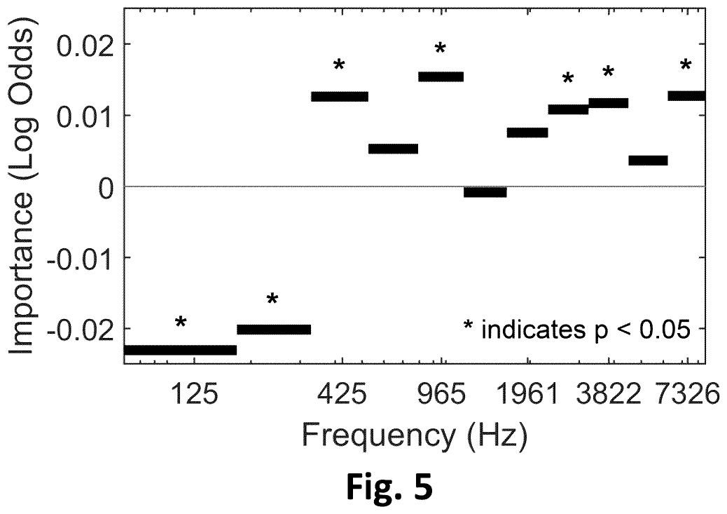

Channel importance is defined as the fixed effect of TMR within each channel on recognition accuracy in a generalized linear model with a logistic link function. This regression accounts for individual differences in overall recognition accuracy in the intercept term, so the relative importance of each channel can be meaningfully compared across listeners. According to at least some aspects of some embodiments, participants (i.e., cochlear implant users) hear and repeat aloud monosyllabic words in the presence of a tone complex masker (see, e.g., FIG. 2 and FIG. 3). The masker is designed to systematically manipulate the availability of speech information within each channel across words, which allows us to measure the impact of masking each channel on word recognition accuracy (see, e.g., FIG. 8). FIG. 5 shows example data using the method provided of estimating importance. Permutation-based split-half reliability indicates that within-participant estimates of importance are reliable (rs=0.75), and examination of fixed-effect correlations and variance inflation factors indicates that estimates of importance are independent across channels.

Channel importance will be quantified by regressing word recognition accuracy against TMR in each channel using a Bayesian generalized linear mixed effects model with a logistic link function, which estimates the impact of masking each channel on the probability of correctly recognizing words. The advantage of this approach is that it estimates the probability distribution of model parameters, which can be used to quantify how (un) likely it is that apparent individual differences could have arisen from sampling variability.

According to some aspects, cochlear implants vary in the allocation of frequencies to channels. The spectrum is partitioned into discrete bins, with edges corresponding to the edge frequencies of channels in each implant. Channel importance will be calculated by summing the importance of frequency bins within each channel's frequency range. The model includes random intercepts and slopes across participants for each channel. These parameters can be used to estimate the importance of the channel for speech recognition when the channel is effectively unmasked log odds at +15 dB TMR), the decline in word recognition accuracy with increasing masker level (the slope), and the TMR at which masker interference overtakes the useful information provided by the channel envelope (the TMR at which log odds is zero). In addition to estimating random effects, Bayesian model fitting allows us to constrain individual random effects to be drawn from a shared group-level distribution. Thus, we can estimate both the group-level distribution of importance for each channel and individual differences in importance within that distribution. We will also use Bayesian regression to estimate the likelihood that speech recognition outcomes without the tone complex masker depend on individual differences in the importance of each channel.

Models with fixed or random individual importance for each frequency bin will be implemented in the Stan language. Leave-one-out cross-validation will be used to select the hypothesis which best explains the data. The advantage of this approach to statistical analysis is that we can estimate the probability distribution of group-level and individual-level model properties. These distributions can be compared to determine the extent to which evidence favors fixed or random importance for each frequency bin, rather than testing for threshold significance criteria.

It should be appreciated that the identification of channel importance has numerous benefits and could provide a number of improvements for users of cochlear implants. It is believed that the information will be able to guide clinicians to maximize the benefit that each and every user of a cochlear implant receives. The information (i.e., channel importance) can provide criteria for clinical decision making and device design that will enable optimization of access to speech cues for all patients. For example, the following include a non-exhaustive list of potential benefits and uses once the channel importance has been determined.

Aim 1: Identify auditory nerve properties that impair encoding of speed cues. Diagnostic measures of channel-specific speech cue access are currently not clinically feasible. Electrophysiological measures of auditory nerve health are clinically feasible, but it is currently unclear how they relate to speech recognition. This aim will establish the link between these measures. It is believed that access to speech cues depends on properties of the auditory nerve, including the strength of its response to stimulation and its speed of recovery from adaptation. The channel importance testing will provide information for modifying clinical processors to avoid stimulating portions of the nerve with poor properties and improves speech recognition. This aim will provide clinical criteria for improving speech recognition by deactivating channels based on electrophysiological measures.

Aim 2: Determine whether bilateral interactions reduce access to speech cues within channels. Many individuals have bilateral cochlear implants, but the benefit of bilateral implantation varies across patients. In this aim, it will be tested whether bilateral asymmetry in speech cue access across ears can impede the listener's ability to understand speech when listening with both ears. This aim will demonstrate the need to evaluate bilateral interactions in the clinic when deciding whether to deactivate channels.

Aim 3: Examine effects of listening conditions on access to speech cues. Two listening conditions with special relevance to hearing outcomes in patients with cochlear implants are listening in quiet and listening in the presence of competing talkers. This aim will assess access to channel-specific speech cues in these conditions. Speech recognition in quiet is a ubiquitous clinical assessment, so users will test whether they can extract additional channel-specific diagnostic information from these assessments. This information will guide clinical decision making toward channels that are likely to have low contributions to speech recognition without requiring any additional clinical evaluation. Speech recognition in competing speech is one of the most challenging listening conditions to patients with cochlear implants. Users will test whether the channels that listeners rely on change in the presence of competing speech. This information will determine the need to adjust implant signal processing strategies to emphasize specific channels in multi-talker environments.

Aim 4: Identify the electrical stimulus features that convey speech cues. Metrics that predict speech recognition accuracy for hearing aids inform device design and validate clinical fittings, but such metrics are not established for cochlear implants. This aim will compare metrics of speech cue availability as a first step toward developing intelligibility metrics for cochlear implants. Testers will record the electrical output of clinically programmed processors for all stimuli used in previous aims. It is believed that transducing the modulation spectrum of the acoustic input into an electrical pulse train is essential for accurate speech recognition. Identifying which acoustic features need to be transduced, which will guide the design of novel processing strategies that preserve access to speech cues.

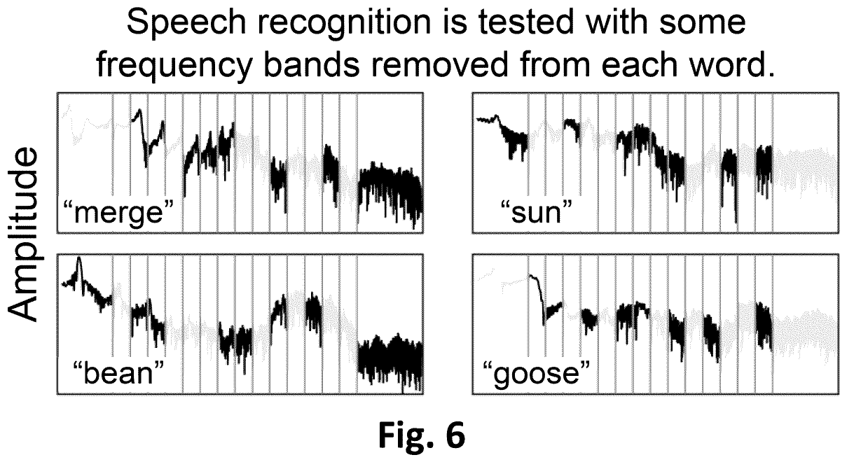

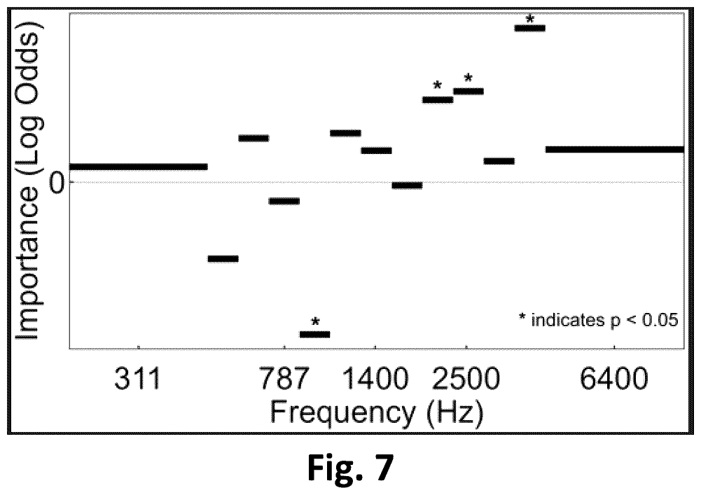

Still additional aspects and/or embodiments are provided for systems and/or methods of the disclosure. According to at least some aspects of some embodiments, cochlear implant users hear and repeat aloud monosyllabic words that have been filtered to remove some frequency bands that correspond to different channels (see, e.g., FIG. 6). Filters are designed to systematically manipulate the presence or absence of speech information within each channel across words, which allows us to measure the impact of removing each channel on word recognition accuracy. Channel importance is defined as the fixed effect of filtering out each channel on recognition accuracy in a generalized linear model with a logistic link function. Filtering is designed to measure the impact of removing each channel on word recognition accuracy (see, e.g., FIG. 7).

“Channel importance” is how much speech information a listener with a cochlear implant can extract from each signal processing channel in their clinical device. Once estimates of channel importance are obtained, there are several ways this information could be used for clinical intervention and/or device modification.

Therefore, as can be appreciated, the disclosure provides methods and/or systems that will provide improvements for users of cochlear implants. The methods and systems provided, as well as obvious variations thereof, will improve the ability of cochlear implant users to be able to hear and speak, which will further improve the use of the cochlear implants.

It should be appreciated that variations and/or changes to any of the components or embodiments that are obvious to those skilled in the art are to be considered a part of the present disclosure. In addition, any of the aspects of any of the embodiments disclosed could be combined in ways not explicitly shown and/or described to provide yet additional embodiments that are part of the disclosure. The disclosure is not to be limited to the embodiments disclosed herein.

1. A method for identifying channel importance of a cochlear implant user, the method comprising:
providing an audio example to the cochlear implant user;
masking one or more channels of the cochlear implant during the audio example, wherein a masker manipulates the availability of information available from the audio example on the channel to obtain a target-to-masker ratio; and
identifying channel importance by determining the fixed effect of the target-to-masker ratio within each channel to a recognition accuracy of the audio example.

2. The method of claim 1, wherein the audio example comprises:
a. a verbal word; or
b. an audio file played on a speaker.

3. The method of claim 1, further comprising having the user identify the content of the audio example to determine the recognition accuracy.

4. The method of claim 3, wherein the identifying of the audio example is done verbally, in writing, or via multiple choice.

5. The method of claim 1, wherein the step of masking the channel comprises masking each channel with a pure tone at a middle of that channel's frequency range.

6. The method of claim 1, further comprising deactivating channels of the cochlear implant for channels having low channel importance.

7. The method of claim 1, wherein the step of masking the channel comprises pseudorandomly selecting a masking level for each channel.

8. A method of improving the performance of a cochlear implant in a user, comprising:
identifying channel importance for an implanted cochlear implant of the user by masking channels of the cochlear implant;
based on the identified channel importance, adjusting the cochlear implant channels.

9. The method of claim 8, wherein the adjusting the cochlear implant channels comprises deactivating channels having low channel importance.

10. The method of claim 8, wherein the step of identifying channel importance comprises:
providing an audio example to the cochlear implant user;
masking one or more channels of the cochlear implant during the audio example, wherein a masker manipulates the availability of information available from the audio example on the channel to obtain a target-to-masker ratio; and
identifying channel importance by determining the fixed effect of the target-to-masker ratio within each channel to an audio recognition accuracy of the audio example.

11. The method of claim 10, wherein the audio example comprises:
a. a verbal word; or
b. an audio file played on a speaker.

12. The method of claim 10, further comprising having the user repeat the audio example to determine the audio recognition accuracy.

13. A method for identifying channel importance of a cochlear implant user, the method comprising:
providing an audio example to the cochlear implant user;
filtering one or more frequency bands that correspond to one or more channels of the cochlear implant; and
identifying channel importance by determining which of the one or more filtered channels affects the cochlear implant user's hearing.

14. The method of claim 13, wherein the filtering one or more frequency bands comprises manipulating a presence of speech information within a channel associated with the audio example.

15. The method of claim 13, wherein the filtering one or more frequency bands comprises manipulating an absence of speech information within a channel associated with the audio example.

16. The method of claim 13, wherein the identifying channel importance comprising a fixed effect of filtering out each channel on recognition accuracy in a generalized linear model with a logistic link function.

17. The method of claim 13, wherein filtering one or more frequency bands measures an impact of removing each channel on word recognition accuracy.

18. A method for identifying channel importance of a cochlear implant user, the method comprising:
providing an audio example to the cochlear implant user;
manipulating one or more channels of the cochlear implant during the audio example; and
identifying channel importance by determining which of the one or more manipulated channels affects the cochlear implant user's hearing.

19. The method of claim 18, wherein the step of manipulating the one or more channels of the cochlear implant comprises masking one or more channels of the cochlear implant during the audio example, wherein a masker manipulates the availability of information available from the audio example on the channel to obtain a target-to-masker ratio.

20. The method of claim 18, wherein the step of manipulating the one or more channels of the cochlear implant comprises filtering one or more frequency bands that correspond to one or more channels of the cochlear implant.
